(19) United States
(12) Patent Application Publication
(10) Pub. No.: US 2008/0214912 A1
(43) Pub. Date: Sep. 4, 2008

(54) Blood Glucose Monitoring System And Method

(75) Inventor: Gerald G. Cano, Export, PA (US)

Correspondence Address: THE WEBB LAW FIRM, P.C., 700 KOPPERS BUILDING, 436 SEVENTH AVENUE, PITTSBURGH, PA, 15219, US

(73) Assignee: GLUCOSE SENSING TECHNOLOGIES, LLC, Pittsburgh, PA (US)

(21) Appl. No.: 11/972,190

(22) Filed: Jan. 10, 2008

Related U.S. Application Data
(60) Provisional application No. 60/879,745, filed on Jan. 10, 2007.

(51) Int. Cl. A61B 5/00; A61B 5/05

(52) U.S. Cl. 600/316; 600/309; 600/407

(57) ABSTRACT

An optrode for insertion into a blood vessel can include at least one fiber optic, a sheath enclosing at least part of the fiber optic and sensor material in optical communication with one end of the fiber optic. The sensor material is operative for diffracting electromagnetic waves received from the fiber optic and is responsive to changes in an amount of glucose in contact therewith for changing a diffraction of the electromagnetic waves as a function thereof. A membrane covering a distal end of the sheath passes to the sensor material glucose from blood in contact with the side of the membrane opposite the sensor material. Electromagnetic waves input into the fiber optic cable are diffracted by the sensor material. A concentration of glucose in the blood can be estimated from the amount of diffraction of the electromagnetic waves. A signal can be output related to the estimated glucose concentration.

### CROSS REFERENCE TO RELATED APPLICATION

This application claims priority from U.S. provisional patent application No. 60/879,745, filed Jan. 10, 2007, which is incorporated herein by reference.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant Number 1 R43 DK067788-01A2 awarded by Department of Health and Human Services—National Institutes of Health—National Institute of Diabetes and Digestive and Kidney Diseases.

### BACKGROUND OF THE INVENTION

1. Field of the Invention

This invention relates to glucose monitoring and, more particularly, to continuous glucose monitoring of the critically ill in an institutional setting.

2. Description of Related Art

Currently in the United States, millions of people have diabetes, that is, they suffer from hyperglycemia (high blood glucose concentration). Many more millions are classified as “pre-diabetic.” People with diabetes, due to its long-term affects, already have a propensity for acute events—heart attack, stroke, kidney failure, neuropathy, limb amputation, etc.

The surge in the number of people who suffer from diabetes has brought about intense interest in improved means to measure the concentration of glucose found in blood. Desired characteristics associated with such improved means to measure include: 1) reduced time to determine, 2) non-invasive or minimally invasive, and 3) continuous. The first, reduced time, is usually associated with the time required for a small portable meter to determine concentration from a small blood sample obtained immediately before by lancing a finger tip or some other part of the body. The second, non-invasive or minimally invasive, is usually associated with some indirect means of measuring blood glucose. Current indirect means have not been accepted due to accuracy or confusion over clinical clarity. Because of the nature of such indirect means, they offer the prospect of continuous or near continuous. In each situation, it is recognized that the users, people with diabetes, are ambulatory and must measure their glucose frequently as they go about activities associated with their daily lives.

However, in recent years, a new medical situation involving elevated blood glucose has been identified. Specifically, hyperglycemia and insulin resistance are common in non-diabetic patients who have had an acute event and are typically treated in the ICU. The term “acute event” includes surgery, heart attack, stroke, recent amputation, organ transplant, etc. In fact, hyperglycemia resulting from the stress of an acute event (>8.0 mmol/L or, equivalently, >145 mg/dL) occurs in over 73% of non-diabetic patients following surgery (Thomas et al., BMC Nephrology (2000) 1:1). The deleterious effects of stress induced hyperglycemia include the following:
1. During heart attack or myocardial infarction (MI), hyperglycemia is associated with increased levels of inflammatory markers, enhanced expression of cytotoxic T-cells, and reduced expression of T-cells, which are implicated in limiting the immune process. An increased inflammatory immune process seems a likely mechanism linking acute hyperglycemia to poor cardiac outcome in MI patients (Marfella et al., Diabetes Care. 2003 Nov. 26 (11):3129-35).
2. Admission plasma glucose, even after adjustment on HbA(1c), is a prognostic factor associated with mortality after acute MI. Acute rather than the chronic pre-existing glycometabolic state accounts for the prognosis after acute myocardial infarction (Hadjadj et al., Diabet Med. 2004 April; 21(4):305-10).
3. It is known that ‘time is brain’, and only early therapies in acute stroke have been effective, like thrombolysis within the first 3 hours, and useful neuroprotective drugs are searched for that probably would be effective only with their very early administration. General care (respiratory and cardiac care, fluid and metabolic management, especially blood glucose and blood pressure control, early treatment of hyperthermia, and prevention and treatment of neurological and systemic complications) in acute stroke patients is essential and must already start in the prehospital setting and continue at the patient's arrival to a hospital in the emergency room and in the stroke unit. A review of published studies analyzing the influence of general care on stroke outcome and the personal experience from observational studies show that glucose levels >8 mmol/l (>145 mg/dL) have been found to be predictive of a poor prognosis after correcting for age, stroke severity, and stroke subtype. Although a clinical trial of glucose-insulin-potassium infusions is ongoing, increased plasma glucose levels should be treated (Diez-Tejedor et al., Cerebrovasc Dis. 2004; 17 Suppl 1:130-7).
4. Hyperglycemia is associated with an increased risk for allograft rejection. This is consistent with similar findings in patients with diabetes (Thomas et al., BMC Nephrol. 2000 Oct. 4; 1(1):1).

There is increasing evidence that maintaining normoglycemia and treatment with insulin, particularly in patients without pre-existing diabetes, helps to limit organ damage after surgery, myocardial infarction, stroke, traumatic brain injury and other conditions, even though these conditions may be accompanied by insulin resistance. Specifically, insulin is credited with the following potentially beneficial actions in the critically ill patient (Groeneveld et al., Critical Care, April 2002 Vol 6 No 2):
1. less (stress) hyperglycemia by “overcoming” insulin resistance, and therefore better antimicrobial defense and wound healing;
2. stimulation of glucose uptake/glycolysis, pyruvate dehydrogenase and energy production;
3. anti-inflammatory properties, such as less oxygen radical formation;
4. suppression of insulin-like growth factor (IGF)-1-binding protein, increased IGF-1;
5. increased muscle protein synthesis;
6. inhibition of apoptosis and promoting repair of damaged tissue;
7. promotion of ischemic preconditioning; and
8. less ischemia/reperfusion damage.

The literature now suggests that intensive insulin therapy to maintain blood glucose at or below about 6 mmols/liter (110 mg per deciliter) reduces morbidity and mortality among critically ill patients in the surgical intensive care unit (Van den Berghe et al., N Engl J Med, Vol. 345, No. 19, Nov. 8, 2001).

This evolving body of information that associates stress-induced hyperglycemia with poor outcome, and, conversely, TGC with improved outcome, has produced an increasing clinical demand for continuous glucose monitoring of the acute patient. Such monitoring must accurately provide a clinically acceptable glucose concentration measurement in a way that does not burden the already over-worked ICU staff. Up to this point, most of the effort and innovation in glucose monitoring has focused on the needs of the diabetic patient.

Technology related to glucose monitoring is segmented by sensor, over all system and method. Obviously, the system is determined by the type and placement of the sensor. The method is generally determined by the type of sensor, one that directly measures glucose or one that provides an indirect measurement: direct measurement is usually invasive; indirect is non-invasive.

U.S. Pat. No. 5,000,901 discloses a manufacturing method and use method for a single fiber optic physiologic probe. The manufacturing method identifies limited polymers suitable for the film sensor. The use method involves contacting a fluid with the sensor, using the fiber optic to illuminate the film with one set of light wavelengths and receiving an emitted different set of light wavelengths from the film, a characteristic indicative of fluorescence. The probe is inserted into the bloodstream through a catheter.

U.S. Pat. No. 5,001,054 discloses a method for monitoring the glucose level in a body fluid using an apparatus which includes a conjugate of glucose oxidase and a fluorescent dye coated onto an optical fiber in contact with the body fluid, a source of excitation light and a fluorescence emission detector. The method requires contacting the body fluid.

U.S. Pat. No. 5,605,152 discloses an in vivo sensor (inserted into interstitial fluid) of one or more optical fiber electrodes mounted within a semipermeable probe housing designed for differential diffusion of glucose and oxygen. The optical fiber is coated with an enzyme such as glucose oxidase for catalyzing glucose in the presence of oxygen (O₂) to produce gluconic acid and hydrogen peroxide. An oxygen sensitive coating such as fluorescent dye is positioned on the optrode close to the enzymatic reaction. Due to issues associated with system inaccuracies, a reference optrode is also used in close proximity to the enzymatic reaction. Monitoring of the fluorescent activity (light brightness at a specific wavelength) provides an indication of oxygen depletion as a result of the reaction and thus the glucose concentration level. The semipermeable membrane is designed to ensure that the reaction proceeds with a stoichiometric excess of oxygen.

U.S. Pat. No. 5,628,310 discloses an apparatus and method to enable minimally invasive transdermal measurements of the fluorescence lifetime of an implanted element without reagent consumption and not requiring painful blood sampling. The monitoring apparatus displays the quantity of a selected substance present in the skin and stores the data in memory. The stored information can be transmitted via modem, or antenna, to a master station for diagnostic purposes or clinical evaluation. The procedure is completely non-invasive after one implant.

U.S. Pat. Nos. 6,002,954; 6,011,984; 6,750,311 and 6,804,544 each disclose a method for determining the concentration of biological levels of polyhydroxilated compounds such as glucose. The in vivo method uses an implantable biocompatible amplification system that includes an analyte transducer immobilized in a polymeric matrix. The transducer changes in dimension in accordance with glucose concentration change. The dimensional change alters the intensity of light received by a photoreceptor. When interrogated by an external system, the amplification system produces a signal capable of detection external to the skin. Quantification of the analyte is achieved by measurement of the emitted signal. The system components, the implanted sensor processing circuitry and the external interrogator/receiver, are determined by the conversion of a light intensity signal to an electrical signal that is then transmitted through the skin to the external system processor.

U.S. Pat. Nos. 6,201,980 and 6,480,730 each disclose an in vivo chemical sensor, permanently implanted, for medical applications that selectively measures an analyte with an expandable biocompatible sensor, such as a polymer, that undergoes a dimensional change in the presence of an analyte. The expandable polymer is incorporated into an electronic circuit component that changes the characteristics of an electronic circuit element (for instance a capacitor, when the polymer changes dimension). As the circuit changes its characteristics, an external interrogator transmits a signal transdermally to the transducer, and the analyte concentration is represented by the measured change in the circuit, in this case a change in the tuned frequency of the circuit.

U.S. Pat. No. 6,224,550 discloses a method for monitoring changes in the level of an osmotically active component such as glucose or lactic acid. The in vivo sensing device is an implanted membrane that responds to the back and forth osmotic movement of the analyte. The response is recorded by a receiver outside the body.

U.S. Pat. No. 6,368,274 discloses a reusable in vivo sensor site for use with a replaceable, long-term implantable in vivo analyte sensor. A particular application is that of a reusable glucose sensor site described for use with replaceable, long-term glucose sensors. The site housing is formed to have an interior cavity with an opening and a conduit that is connected to the opening of the interior cavity to provide access to the interior cavity, wherein the site housing material is selected to promote tissue in-growth and vascularization, and yet free of tissue ingress. The housing material permits the analyte to pass through the site housing to the interior cavity to permit measurement by the replaceable analyte sensor. Essentially, the patent discusses a means for monitoring an analyte, but does not disclose a specific sensor.

U.S. Pat. No. 6,475,750 discloses an in vivo biosensor system, including a sensor, for measuring glucose in blood. The sensor is a confined volume that contains a hydrogel that shrinks or swells relative to glucose concentration, thus changing pressure within the confined volume. Volume change is controlled by an immobilized glucose-binding molecule such as concanavalin A (Con A) and an immobilized hexose saccharide such as a-D mamiopyranoside. The immobilized hexose saccharide competitively binds with free glucose to the glucose binding molecules, changing the crosslinks in the hydrogel that change hydrogel swelling tendency. The system is not subject to problems with oxygen limitations associated with certain other sensors using glucose oxidase. Readings from the sensor are telemetered from the transducer to a receiver for processing and interpretation.

The most commonly used glucose sensors for in vivo monitoring are electrochemical sensors using the amperometric technique, because they do offer the possibility for a linear calibration curve in a significant portion of the blood glucose concentration range. Generally, these sensors are described as “minimally invasive” as they only penetrate the skin to contact the interstitial fluid under the skin. In the amperometric method, an electrode is used which produces a current proportional to the diffusional flux of hydrogen peroxide (H₂O₂) to the electrode surface, or, alternatively, proportional to the diffusional flux of oxygen (O₂) to the electrode surface. A membrane layer containing immobilized glucose oxidase (GOD) surrounds the electrode. The glucose reaction catalyzed by GOD produces hydrogen peroxide and consumes oxygen. An increase in the surrounding glucose concentration should increase the diffusional flux of glucose into the membrane and increase the reaction rate within the membrane. The increase in reaction rate in turn should increase the local hydrogen peroxide concentration and decrease the local oxygen concentration within the membrane. This should lead to an increase in the current detected by a hydrogen peroxide-based electrode sensor, or a decrease in current as detected by an oxygen-based electrode sensor. The latter approach, based on detecting the oxygen flux, also requires a second oxygen-based electrode sensor located in a hydrogel without the GOD enzyme. This second electrode is used as a reference.

Amperometric sensors must overcome several hurdles before they will ever be useful for commercial in vivo monitoring. Current glucose sensor designs appear unlikely to solve these difficult problems in the near future. The first hurdle arises from electrochemical interference. The analyte (whether hydrogen peroxide or oxygen) must be the only species present which produces a current at the electrode. Hence, for both oxygen-based and hydrogen peroxide-based glucose sensors, an inner membrane must be used which is permeable to the analyte but impermeable to endogenous interferents. This is a difficult goal to achieve due to the heavily “contaminated” nature of blood. Secondly, for the hydrogen peroxide-based sensor, mass transfer coefficients for diffusion of glucose and oxygen into the membrane containing GOD must not change with time due to an adsorbed layer. Thirdly, for both types of amperometric sensors, GOD must not deactivate with time. In clinical studies of the hydrogen peroxide-based sensor, decay in sensitivity over the implant period was observed, a phenomenon that could not be explained by blockage of the sensor surface by protein. One possible explanation for the loss of sensitivity is hydrogen peroxide mediated GOD deactivation. For the oxygen-based sensor, this can be avoided by co-immobilizing catalase with GOD, because catalase consumes hydrogen peroxide. Fourthly, a shortage of oxygen relative to glucose can place an upper limit on the biosensor's ability to measure glucose levels. This problem is called the “oxygen deficit”. These types of sensors are discussed in U.S. Pat. Nos. 4,680,268; 4,721,667; 5,356,786; and 6,881,551.

U.S. Pat. No. 5,469,846 discusses a non-enzymatic implantable in vivo glucose sensor that must be energized by a DC voltage and that produces a DC current whose magnitude is directly related to glucose concentration. The electrode is constructed of a non-reactive tin oxide semiconductor. The device also uses a variable voltage to enable the electrode to be self-cleaning.

U.S. Pat. Nos. 5,854,078; 5,898,004; 6,187,599; 6,544,800; 6,753,191; and 7,105,352 discuss colorimetric sensors formed from polymerized crystalline colloidal array (PCCA) materials. Additionally, they disclose the combination of the sensor with fiber optics to create optrodes for sensing various analytes. The patents state that the PCCA can be attached to the end of a fiber optic and that light channeled down the fiber will be reflected back into the fiber to carry information on the PCCA volume, and thus the analyte concentration. This embodiment is effective since a spectrometer can be used to detect very small changes in diffracted wavelength. However, response time is a function of the sensor material and analyte concentration. Such an optrode can be easily miniaturized since only a small piece, approximately 1 μm³ or smaller, of the PCCA is needed for the sensor. However, these patents do not functionalize it for in vivo use, that is, they do not address placement, biocompatibility, protein deposition, and retardation of microorganism growth. Moreover, they do not provide details for electronics needed to analyze the sensor response or how to use the information. Furthermore, they do not differentiate a functionalized optrode for colorimetric measurement from that of an optrode for fluorescence measurement.

U.S. Pat. No. 6,835,553 discloses a battery powered photometric glucose measurement system using a glucose-sensitive gel. The sensor component of the system is configured to reside in vivo in body fluids. As disclosed, the system utilizes a hydrogel filament sensor sensitive to the analyte of interest, glucose. The hydrogel sensor undergoes a change in its displacement volume; a photometric displacement transducer quantifiably detects changes in displacement volume and represents such by a change in light intensity. An internal telemeter sends radio data to an external receiver. The signal sounds an alarm to notify the patient that the analyte level is outside of the predetermined range. Additionally, the patent discloses a method for determining the concentration of free analyte using the glucose measurement system. The method does not discuss placement of the sensor component in vivo.

U.S. Pat. No. 6,946,086 discloses a colorimetric PCCA sensor comprising an ordered array of colloidal particles locked in place in polymerized poly(ethylene glycol).

U.S. Pat. Nos. 6,689,316; 2006/0063038; and 2006/0166350 disclose a holographic sensor, method for its production, and method for analyte detection using the sensor. Essentially, the sensor responds to a stimulus with a change in at least one optical characteristic. Typically, when measuring the concentration of an analyte, this optical characteristic is a change in color due to diffraction, thus providing a colorimetric response. The method of detection is quite general in that it merely specifies contacting the sample with the sensor. U.S. Patent document 2007/0153343 discloses a sensor and a method for use. The sensor comprises a medium and, disposed therein, a hologram, wherein an optical characteristic of the hologram changes as a result of a variation of a physical property of the medium, and wherein the hologram is formed as a non-planar mirror.

U.S. Pat. No. 7,149,562 discloses a needle with fiber optic capability for application in spectrophotometric analysis comprising a needle body with an open tip and side radiation ports. A fiber optic element is used to carry electromagnetic radiation to the open tip area for transmission to a target area. Fiber optic bundles in communication with the radiation ports transmit backscattered radiation to a light detector or a sensor for spectrophotometric analysis. Additionally, it discloses a monitoring device including the needle with fiber optic capability and components needed for creating electromagnetic radiation and a means for sensing backscattered radiation. This means for sensing the backscattered radiation is not proximal to the tip or the radiation ports, but at the end of the needle opposite the tip.

As can be seen, the prior art discloses various sensor technologies applicable for measuring glucose concentration in a fluid or liquid, such as, without limitation, hydrogel sensors. Fundamentally, they all function by changing dimensions. The dimension change is then manifest by various phenomena, including modification of optical properties. Those that depend on a measurement of brightness are less robust than those that depend on a measurement of wavelength, that is, measuring brightness at a given wavelength is less robust than measuring a change in wavelength.

Additionally, several systems are meant for implantation to enable in vivo measurement of glucose in a body fluid. Such implantation is intended for extended use by ambulatory diabetic patients. They are not intended for brief periods of a few days to perhaps a week. Additionally, several systems are electrically powered; batteries eventually fail and also have safety and biocompatibility issues. Additionally, implanted systems are expensive, involve minor surgery for implantation, and can only be used on one patient, even if it is possible to surgically remove them.

U.S. Pat. Nos. 6,846,288 and 7,254,429 disclose continuous non-invasive means and systems to measure glucose concentration by indirect means.

U.S. Pat. No. 6,846,288 discloses the use of photoacoustics which involves directing light at a particular wavelength to a region of tissue, transmitting ultrasound so that it is incident on the region; measuring at least one effect of the change on the incident ultrasound; using the measured at least one effect to determine an absorption coefficient for the radiation in the region; and using the determined absorption coefficient to determine concentration of the component in the region.

U.S. Pat. No. 7,254,429 discloses a low coherence interferometer to non-invasively monitor the concentration of glucose in blood by shining a light over a surface area of human or animal tissue, continuously scanning the light over a two-dimensional area of the surface, collecting the reflected light from within the tissue and constructively interfering this reflected light with light reflected along a reference path to scan the tissue in depth. Since the reflection spectrum is sensitive to glucose concentration at particular wavelengths, measurement and analysis of the reflected light provides a measure of the level of glucose in the blood. In each case, with these indirect means, it is unclear as to the relationship of the measurement to the actual blood glucose concentration.

Accordingly, there exists a need for a continuous glucose monitoring system and sensor suitable for the needs of acute patients while in an ICU. The sensor should be a temporary in vivo component that specifically measures blood glucose concentration. It should be biocompatible and retard growth of microorganisms when a portion is positioned percutaneously within a blood vessel. The sensor should be easily positioned in the blood vessel of a patient by ICU staff who are typically trained to insert catheters into a blood vessel. The sensor should be easily removable at the end of monitoring. The sensor and system should not elevate the likelihood of electrical shock. The sensor should be robust. The system should reliably interpret the sensor output, accurately indicate blood glucose concentration, and alarm when the concentration is outside of a programmed range.

### SUMMARY OF THE INVENTION

Disclosed is a glucose monitoring system for the intensive care unit that produces a continuously updated numerical readout of blood glucose concentration and automatic alarm of out-of-range blood glucose concentration. The system comprises colorimetric glucose sensors and electronics for continuously monitoring glucose concentration level in a body fluid, such as blood.

Also disclosed is a glucose sensor having one or more optical fibers to form an optrode for monitoring glucose concentration, wherein the optrode is a compact sensor probe adapted for in vivo implantation. The optrode includes a hydrogel colorimetric sensor material affixed to the tip of the optrode whose color is representative of the glucose concentration in the blood of the patient.

The glucose monitoring system includes the following:

1. A percutaneous vascular access means such as a central or venous access catheter or vascular port that facilitate insertion of devices into the vasculature.

2. A calorimetric optrode that is inserted percutaneously into a blood vessel via the vascular access means to continuously quantify blood glucose concentration.

3. An appropriate connection linking the optrode to a monitor unit, the connection facilitating the transmission of light to the colorimetric sensor material at the tip of the optrode from the monitor unit and the transmission of reflected light from the sensor back to the monitor unit.

4. A monitor unit comprised of electronics to service the optrode and perform various functions associated with system operation in the ICU environment, such as, without limitation:
a. a “white” light source or near “white” light source focused such that its light is transmitted through the fiber optic connection linking to the colorimetric optrode;
b. a color sensitive receiver to recover and determine the wavelength of light reflected back from the optrode;
c. a microprocessor to read the output of the color sensitive receiver and to interpret the wavelength received by the color sensitive receiver from the optrode;
d. software to translate the received wavelength to a glucose concentration and control displays and alarms;
e. an alpha-numeric display to present the glucose concentration;
f. an alarm (audible and/or optical) operated by the microprocessor to notify personnel that blood glucose concentration exceeds the normal range;
g. memory to store periodic readings of glucose concentration for trend analysis; and
h. a key pad or other information entry device to program and control the system.

The design of the optrode is such that it can be compact and cost-efficient so that it can be treated as disposable.

Use of the system should not require ICU staff to learn new clinical skills or impose a burden on their time. ICU staff are skilled on the placement of vascular access devices such as catheters and vascular ports. Placement typically involves 1) percutaneous insertion of a “guide” needle into a blood vessel inserted at an angle oblique to the surface of the skin, 2) placement of a tubular “expander” over the guide needle to expand the channel created by the guide needle to facilitate placement of the catheter, 3) removal of the guide needle, 4) placement of the catheter over the expander, and 4) removal of the expander. The external portion of the catheter (that external to the surface of the skin) is typically taped to the skin to maintain positioning.

Implementation of the calorimetric optrode for glucose concentration measurement is facilitated by use of a calorimetric sensor material that responds to changes of glucose concentration within a body fluid. U.S. Pat. Nos. 5,854,078; 5,898,004; 6,187,599; 6,544,800; 6,753,191; and 7,105,352 disclose polymerized crystalline colloidal array (PCCA) material and array sensors. U.S. Pat. Nos. 6,689,316; 2006/0063038; 2006/0166350; and 2007/0153343 disclose holographic materials and sensors.

The optrode is inserted into the blood vessel through the catheter so that the tip of the optrode containing the calorimetric sensor material extends beyond the catheter into the blood vessel. The optrode may be taped in place to the skin or fixed to the catheter via some fixation means.

The optrode can minimally be a single fiber optic with calorimetric sensor material affixed to the tip. In such a configuration, the same fiber optic transmits “white” light to the sensor material at the tip and also transmits reflected light from the sensor material back toward the monitor unit. However, there is a technical cost associated with using one fiber optic. Namely, the complexity of the optics in the monitor must accommodate white light that is entering and reflected light (representative of the glucose concentration) that is exiting the same end of the single fiber optic. For example, see U.S. Pat. No. 4,577,109. However, multiple fiber optics can be used so that “white” light is transmitted to the sensor material via one or one group of fiber optics and reflected light is returned via another fiber optic or group of fiber optics. The technical cost of multiple optic fibers is an increase in the diameter of the optrode.

The design of the optrode desirably takes into account the application, namely, measuring glucose in blood via percutaneous insertion into a blood vessel via a catheter or equivalent device. Accordingly, infection, biocompatibility, and protein deposition are concerns that are desirably considered. The optrode desirably is flexible and has a small diameter (˜1 mm or less). The fiber optic or multiple fiber optics that make up the optrode are desirably coated or enclosed in thin-walled sheathing that is biocompatible and that retards or prevents growth of microorganisms.

The calorimetric sensor material can be fixed by various means to the tip of the fiber optic(s) that extends into the blood vessel. Such material includes those known in the art, including hydrogel PCCA sensors and hydrogel holographic sensors. Fixing the material can be accomplished by various means including epoxy.

Additionally, the sensor material is desirably protected from protein deposition. Protection to prevent deposition can include slightly recessing the sensor material from the tip of the optrode and covering with, for instance, a dialysis membrane. Particularly, in the case of an optrode comprising multiple fiber optics, body fluids can progress up the optrode and out of the body in the spaces between the fiber optics; thus these spaces are desirably filled with a soft flexible filler material such as polyurethane or other similar biocompatible polymer.

The optrode connects with the monitor unit. The connection can be accomplished through an extension of the optic fibers of the optrode to a length of perhaps three or four feet, a length that conveniently allows for positioning of the tip of the optrode with the sensor material in the patient and connection of the other end of the optic fibers to the monitor that is either mounted on a pole or in a rack. The end of the fiber optics outside the patient can include an appropriate connector that mates with the connector on the monitor.

The monitor unit desirably supports numerous functions to be effective in tight glycemic control without elevating the workload of ICU personnel. The monitor unit desirably is relatively small so that it can be mounted on a typical pole used in the ICU for holding different monitoring and dispensing devices. The monitor desirably is electrically powered, either via an electrical outlet or a battery. The monitor desirably includes an on/off switch to control power applied to internal electronic components. The monitor desirably has a digital display, minimally displaying in a continuous fashion the blood glucose concentration of the patient. The monitor desirably houses all electronics and circuitry needed to properly operate the optrode. This includes a “white” light source, here meaning a light source that emits all wavelengths in the at least the visible spectrum at the same amplitude. The monitor desirably also includes a spectrometer that measures the amplitude of light wavelengths in the visible spectrum. It should also include a microprocessor with memory to perform certain processing functions, logic functions, control functions and data storage functions. Processing functions may include converting the output of the spectrometer to blood glucose concentration. Logic functions may include comparisons of blood glucose to certain thresholds. Control functions may include activating a digital display and activating an alarm when blood glucose concentration is outside predetermined limits. Data storage may include a historical record of patient blood glucose concentrations during the monitoring period.

More specifically, the invention is a blood glucose monitoring system comprising: at least one fiber optic cable; a biocompatible sheath enclosing at least part of the at least one fiber optic cable; sensor material supported at one end of the sheath in optical communication with one end of the at least one fiber optic cable, the sensor material operative for diffracting electromagnetic waves received from the at least one fiber optic cable, the sensor material responsive to changes in an amount of glucose in contact therewith for changing a diffraction of the electromagnetic waves as a function thereof; and a biocompatible membrane covering the one end of the sheath, the biocompatible membrane in contact with the sensor material and operative for passing to the sensor material glucose from blood in contact with the side of the membrane opposite the sensor material, while avoiding the passage of one or more blood proteins to the sensor material.

The system can further include means for determining a concentration of glucose in the blood, said means responsive to the diffracted electromagnetic waves for determining as a function thereof the concentration of glucose in the blood and for outputting a signal related thereto.

The system can further include another fiber optic cable enclosed at least in part by the sheath, wherein one fiber optic cable is operative for delivering the electromagnetic waves to the sensor material and the other fiber optic cable is operative for delivering the diffracted electromagnetic waves to the means for determining.

At least one fiber optic cable can deliver the electromagnetic waves to the sensor material and delivers the diffracted electromagnetic waves to the means for determining.

The electromagnetic waves can comprise visible light.

The sensor material can be comprised of either polymerized crystalline colloidal array (PCCA) material or holographic material. The PCCA material and the holographic material are each comprised of a hydrogel, means for diffraction and a molecular recognition agent that expands and contracts the sensor material as a function of the amount of glucose in contact therewith.

The hydrogel of the PCCA material can be one of the following: an acrylamide; purified agarose; N-vinylpyrolidone; methacrylate or hydroxy-ethyl-methacrylate. The hydrogel of the holographic material is one of the following: polyvinyl alcohol; polyvinylpyrrolidone; polyhydroxyethyl acrylate; polyhydroxyethyl methacrylate; polyacrylamides; polymethacrylamides; homopolymers, or copolymers, of polymerisable derivatives of crown ethers; or esters of, or co- or terpolymers of, polyhydroxyethylacrylate, polyhydroxyethylmethacrylate, polymethacrylamide or polyacrylamide.

Desirably, the PCCA material is either acrylamide or hydroxy-ethyl-methacrylate.

The means for diffraction of the PCCA material can be one of the following: self organized colloidal particles or charged colloidal particles selected from the group consisting of colloidal polystyrene, polymethylmethacrylate, silicon dioxide, aluminum oxide, polytetrafluoroethylene and poly N-isopropylacrylamide. The means for diffraction of the holographic material can be silver halide.

The molecular recognition agent of the PCCA material can be one of the following: glucose oxidase; dihydroxides of boron, barium, calcium, magnesium, and strontium; boronic acid; phenylboronic acid; p-nitrophenylboronic acid; 4-methoxyphenylboronic acid; alpha-naphthylboronic acid; 4-aminomethyl-2-N,N′-dimethylaminomethylphenylboronic acid; 3-fluoro-4-aminophenylboronic acid; 2-fluoro-5-aminophenylboronic acid, or boronic acid derivatives having a pKₐ value less than 7. The molecular recognition agent of the holographic material can be one of the following: 2-(((4-(acrylamidomethyl)phenylamino)methyl)phenylboronic acid; 2-((3-methacrylamidopropylamino)methyl)phenylboronic acid; acrylamido-phenylboronic acid; 2-acrylamido-phenylboronic acid; 3-acrylamido-phenylboronic acid; 3-acrylamido-6-fluoro-phenylboronic acid; amino-fluoro-phenylboronic acid; 4-amino 3-fluoro-phenylboronic acid; or 5-amino 2-fluoro-phenylboronic acid.

The means for determining can be operative for periodically determining a concentration of glucose in the blood.

The sheath can be made from a polymer having a coating that avoids the growth of microorganisms.

The invention is also a method of measuring blood glucose concentration comprising: (a) inserting a vascular access catheter percutaneously into a blood vessel; (b) inserting through the lumen of the catheter and into a flow of blood in the blood vessel at least the distal end of an optrode comprised of at least one fiber optic cable, a biocompatible sheath enclosing at least part of the at least one fiber optic cable, sensor material supported at the distal end of the sheath in optical communication with one end of the at least one fiber optic cable, the sensor material operative for diffracting electromagnetic waves received from the at least one fiber optic cable, the sensor material responsive to changes in an amount of glucose in contact therewith for changing a diffraction of the electromagnetic waves as a function thereof, and a biocompatible membrane covering the distal end of the sheath, the biocompatible membrane in contact with the sensor material and operative for passing to the sensor material glucose from blood in contact with the side of the membrane opposite the sensor material while avoiding the passage of one or more blood proteins to the sensor material; (c) inputting electromagnetic waves into the at least one fiber optic cable; (d) estimating from the diffraction of the electromagnetic waves input in step (c) a concentration of glucose in the blood; and (e) outputting a signal related to the estimate in step (d).

Desirably, steps (c) and (e) are repeated substantially continuously.

The electromagnetic waves of step (c) can comprise visible white light.

The optrode can include another fiber optic cable enclosed at least in part by the sheath. One fiber optic cable can be operative for delivering the electromagnetic waves to the sensor material while the other fiber optic cable can be operative for delivering the diffracted electromagnetic waves from the sensor material.

The sensor material can be comprised of either polymerized crystalline colloidal array (PCCA) material or holographic material. The PCCA material and the holographic material can each be comprised of a hydrogel, means for diffraction and a molecular recognition agent that expands and contracts the sensor material as a function of the amount.

### BRIEF DESCRIPTION OF DRAWINGS

FIG. 1 is an exemplary chart of glucose concentration versus diffraction blue shift in nm of a calorimetric sensor material useable with the optrodes shown in FIGS. 2 and 3;

FIG. 2 is a first embodiment optrode for colorimetric measurement of body analytes in vivo having a single fiber optic;

FIG. 3 is a second embodiment optrode for calorimetric measurements of body analytes in vivo comprised of two fiber optics;

FIG. 4 is a typical prior art catheter that can be inserted percutaneously into a blood vessel;

FIG. 5 shows the catheter of FIG. 4 with the distal end thereof inserted percutaneously into a lumen of a blood vessel (vein) with a tip of the catheter inside the lumen and with the optrode of FIG. 2 or 3 extending from the tip of the catheter;

FIG. 6 is a block diagram of an exemplary glucose monitor for use with the optrode of FIG. 2 or 3 for detecting light diffracted by the colorimetric sensor material at the tip of said optrode and for determining from the diffracted light a concentration of glucose in the blood; and

FIG. 7 is a matrix of hydrogels, diffraction means and molecular recognition agents that can comprise the polymerized crystalline colloidal array (PCCA) sensor material and the holographic sensor material for the optrodes of FIGS. 2 and 3.

### DETAILED DESCRIPTION OF THE INVENTION

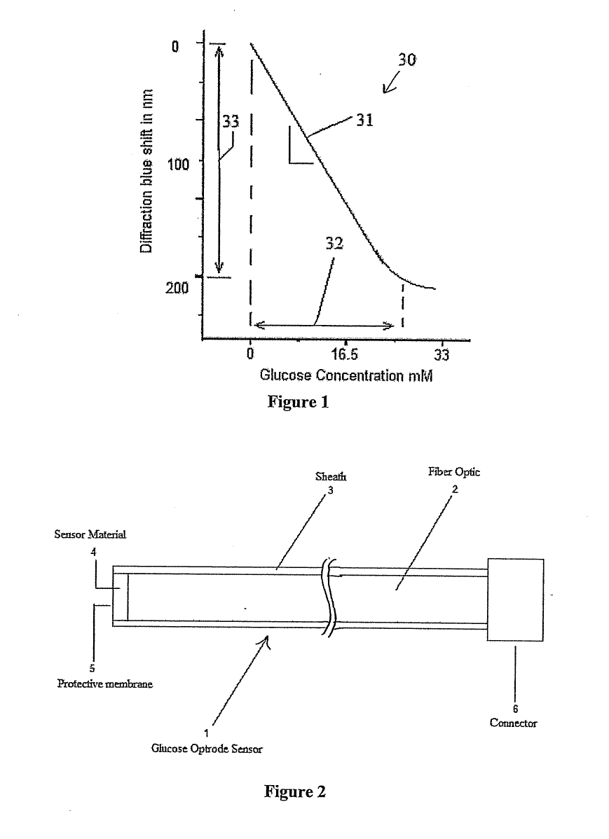

With reference to FIG. 1, a clinically relevant sensitivity curve 30 for a colorimetric glucose sensor material is tailored to an expected glucose concentration range in a particular body fluid, such as blood. Sensitivity curve 30 is suitable for measuring blood glucose concentration when the colorimetric glucose sensor material is immersed in blood. In blood, glucose concentration may possibly vary from 40 mg/dL to 500 mg/dL with normal being in the 80-110 mg/dL range. Sensitivity curve 30 shows a 200 nm wavelength shift 33 over a glucose concentration change 32 from 0 nM to 27.5 mom (0 to 500 mg/dL) glucose concentration. The slope 31 of the curve over the active region of the sensor is defined as:

### Wavelength Shift

### Glucose Concentration Shift

Slope 31 for sensitivity curve 30 is then −07.27 nm/mM (−0.4 nm/(mg/dL)). Slope 31 is negative indicating that the sensor material is at the red end of the visible spectrum at low blood glucose concentration and moves toward the blue end of the visible spectrum as glucose concentration increases. The reflected wavelength produced by the diffraction process decreases as glucose concentration increases. As the sensor material is immersed in blood, it must function in a fluid with 150 mM NaCl at a pH of about 7.4. Such a sensor material can be a polymerized crystalline colloidal array sensor (PCCA) or a holographic sensor as are known in the art. Note the sensor material may be such that the reflected wavelength increases as glucose concentration increases so that the slope would then be positive.

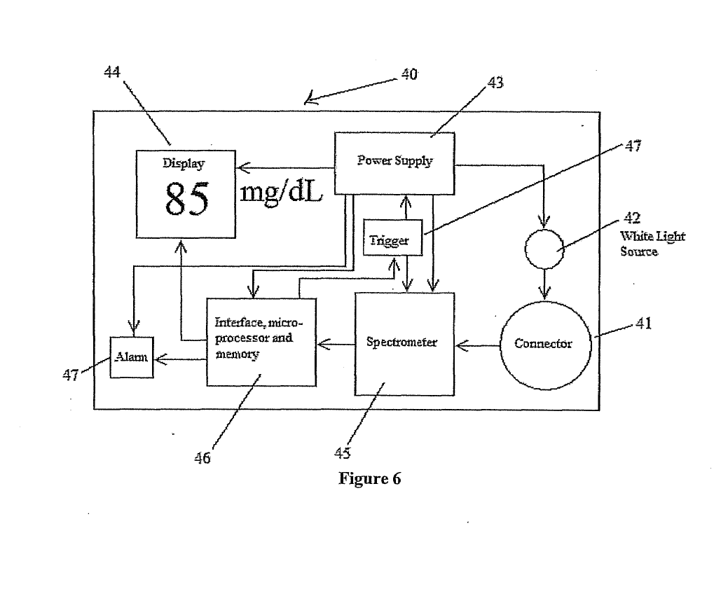

With reference to FIG. 2, a first embodiment optrode 1 includes a single fiber optic wire or cable 2 that transmits electromagnetic waves, desirably in the form of “white” light, from a suitable source thereof (not shown) to colorimetric sensor material 4 at a tip of optrode 1. The “white” light is diffracted by the colorimetric sensor material 4 in the tip of optrode 1 and the wavelength(s) of the diffracted light reflected back along fiber optic 2 represent glucose concentration. The fiber optic 2 is housed in a protective sheath 3 composed of a polymer that retards the growth of microorganisms. Protective sheath 3 and fiber optic 2 can run continuous from the tip or distal end of optrode 1, configured to be inserted into a patient, to a connector 6 that is configured to attach to a connector 41 of a monitor 40 shown in FIG. 6. The tip of optrode 1 is covered by a protective membrane 5 that avoids or prevents deposition of proteins on the surface of the sensor material 4. Such a membrane can be made from a dialysis membrane material.

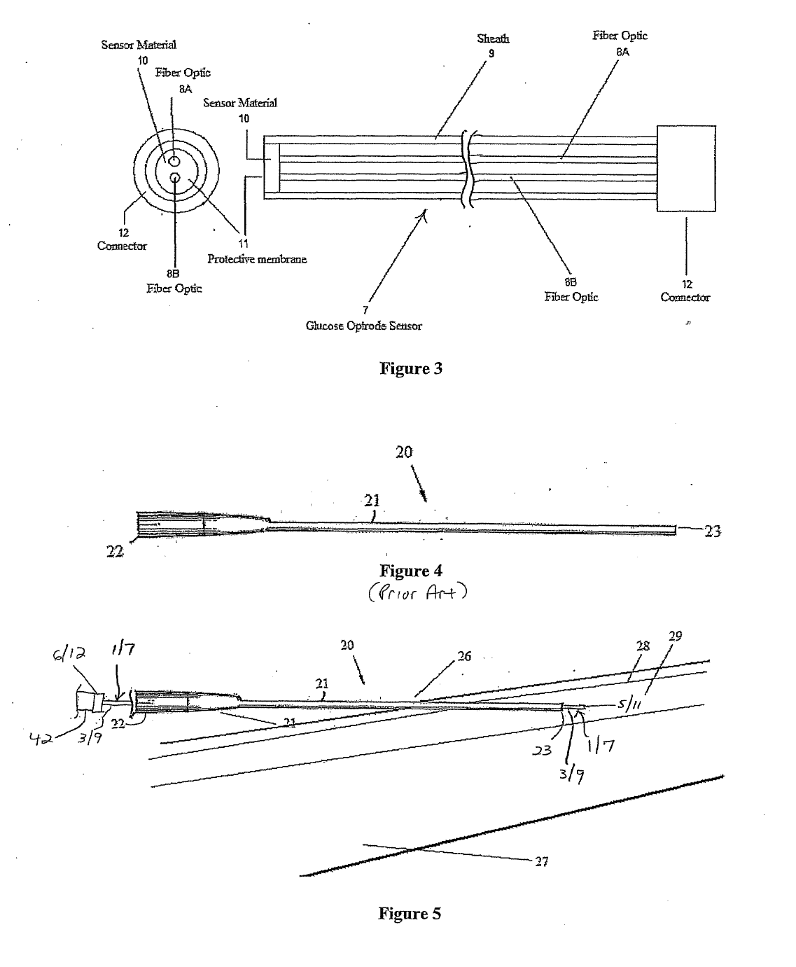

With reference to FIG. 3, a second embodiment optrode 7 includes fiber optics 8A and 8B contained within a sheath 9. Fiber optics 8A and 8B run continuous from the tip or distal end of optrode 7, configured to be inserted into a patient, to a connector 12 of optrode 7 that attaches to connector 41 of monitor 40 shown in FIG. 6. As shown in FIG. 3, optrode 7 comprises, in one preferred form, a pair of optical fibers 8A and 8B mounted within a sheath 9 made of a polymer that is biocompatible and avoids or prevents microbial growth. The two fiber optics 8A and 8B within sheath 9 form a unit long enough to extend conveniently between the patient, where the sensor tip is within a blood vessel, and the externally located monitor 40 shown in FIG. 6 that is typically pole mounted next to the patient.

One fiber optic 8A or 8B transmits electromagnetic waves, desirably in the form of “white” light, from a suitable source thereof (not shown in FIG. 3) to the colloidal array sensor material 10 at the tip of optrode 7. The “white” light is diffracted by calorimetric sensor material 10 in the tip of optrode 7 and the wavelength(s) of the diffracted light reflected back along fiber optic 2 represent glucose concentration. The fiber optics 8A and 8B are housed in protective sheath 9 comprised of a polymer that retards the growth of microorganisms. The tip of optrode 7 is covered by a protective membrane 11 that prevents or retards deposition of proteins on the surface of sensor material 10.

With reference to FIG. 4, a typical catheter 20 has a hollow central channel 21 that extends from the entry port 22 to the tip 23.

In FIG. 5 catheter 20 is shown inserted into the lumen 29 of a blood vessel 28. The tip of optrode 1 or optrode 7 covered by protective membrane 5 or 11 is inserted into entry port 22 and through central channel 21 and out through tip 23 into lumen 29 of blood vessel 28. Connector 6 or connector 12 remains external to catheter 20. In this way, the protected sensor material 4 or sensor material 10 is exposed to glucose in the blood in lumen 29 of blood vessel 28. Thus when the sensor material 4 or sensor material 10 is illuminated by white light, the diffracted color reflected from the sensor material 4 or sensor material 10 is representative of the blood glucose concentration.

Other configurations are possible for the optrode. For instance, the fiber optics can be arranged such that there is a central fiber surrounded by multiple fibers. The central fiber transmits the incident light to the sensor material and the surrounding fibers return the reflected light for analysis.

With reference to FIG. 6, electronics associated with an acute care glucose monitor 40 useable with optrode 1 or 7 includes connector 41 that is configured to mate with connector 6 or 12, respectively. Connector 41 aligns light source 42 with the appropriate fiber optic for transmittal of “white” light to sensor material 4 or 10. Connector 41 also aligns the fiber optic returning the diffracted light with a spectrometer 45 of monitor 40, whereupon the returned diffracted light is received by spectrometer 45.

Spectrometer 45 breaks down the diffracted light from the sensor material 4 or 10 into its component wavelengths that represent blood glucose concentration. The output of spectrometer 45 is transmitted to an interface, microprocessor and memory block 46 where the wavelength is translated to a blood glucose concentration. This concentration is then displayed in digital form on a display 44 by the interface, microprocessor and memory block 46.

Interface, microprocessor and memory block 46 can activate a trigger 47 that detects when the glucose concentration reading is updated on display 44. Trigger 47 then initiates application of power from power supply 43 to “white” light 42 and signals the spectrometer 45 to take another reading. In this manner, a new reading of diffracted light from sensor material 4 or 10 is obtained by spectrometer 45 upon the display of blood glucose concentration from the previous reading on display 44.

Alarm 47 is a circuit that can transmit a signal to activate an alarm (audio and/or visual) either at monitor 40 or remotely at a nurse's station to indicate elevated blood glucose concentration. The level for the alarm is settable and desirably would be above the highest normal blood glucose level 120 mg/dL.

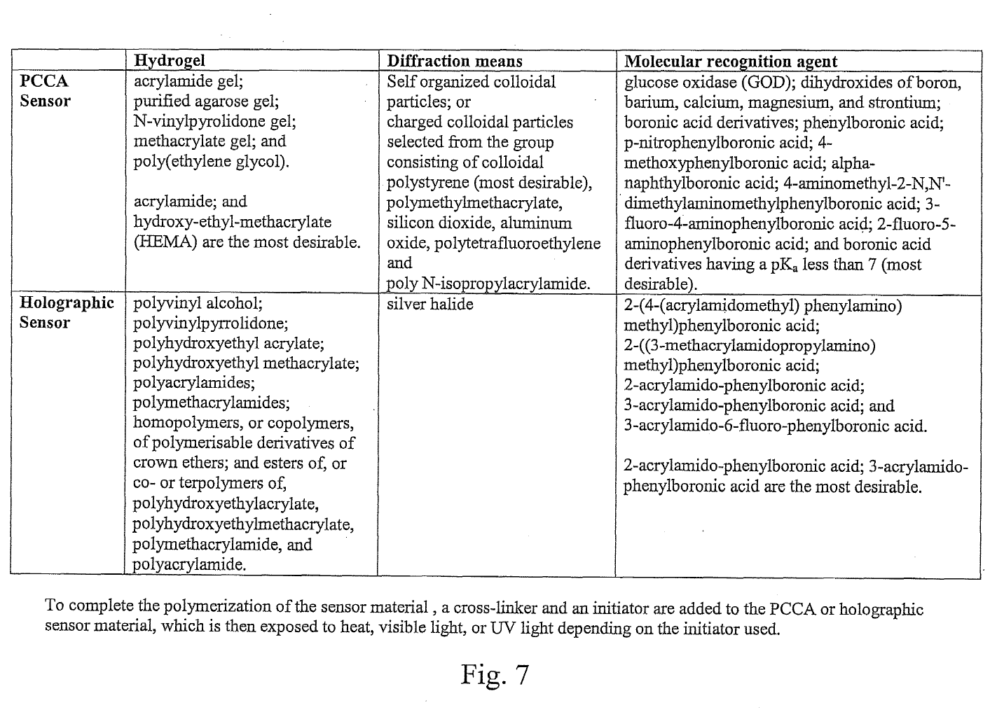

Sensor material 4 or 10 can be either a polymerized crystalline colloidal array (PCCA) sensor material or a holographic sensor material, each of which is comprised of a hydrogel, a diffraction means and a molecular recognition agent, the latter of which causes the entire sensor material to expand and contract as a function of the amount of glucose in contact with the sensor material. A matrix of possible combinations of hydrogels, diffraction means and molecular recognition agents that can comprise a PCCA sensor material and a holographic sensor material is shown in FIG. 7. In FIG. 7, for each sensor, the listed hydrogels, diffraction means and molecular recognition agents can be mixed and matched in any suitable and/or desirable combination.

When boronic acid is the molecular recognition agent of a PCCA sensor, the boronic acid desirably is suitable for use in a fluid which has a pH that is in the range of 7.0 and 7.5. The boronic acid can be an amino-fluoro-phenylboronic acid, a five-amino two-fluoro-phenylboronic acid, a four-amino three-fluoro-phenylboronic acid, an acrylamido-phenylboronic acid, a two-acryamido-phenylboronic acid or a three-acryamido-phenylboronic acid.

The invention has been described with reference to the preferred embodiment. Obvious modifications and alterations will occur to others upon reading and understanding the preceding detailed description. It is intended that the invention be construed as including all such modifications and alterations insofar as they come within the scope of the appended claims or the equivalents thereof.

The invention claimed is:

1. A blood glucose monitoring system comprising:
at least one fiber optic cable;
a biocompatible sheath enclosing at least part of the at least one fiber optic cable;
sensor material supported at one end of the sheath in optical communication with one end of the at least one fiber optic cable, the sensor material operative for diffracting electromagnetic waves received from the at least one fiber optic cable, the sensor material responsive to changes in an amount of glucose in contact therewith for changing a diffraction of the electromagnetic waves as a function thereof; and
a biocompatible membrane covering the one end of the sheath, the biocompatible membrane in contact with the sensor material and operative for passing to the sensor material glucose from blood in contact with the side of the membrane opposite the sensor material while avoiding the passage of one or more blood proteins to the sensor material.

2. The system of claim 1, further including means for determining a concentration glucose in the blood, said means responsive to the diffracted electromagnetic waves for determining as a function thereof the concentration of glucose in the blood and for outputting a signal related thereto.

3. The system of claim 2, further including another fiber optic cable enclosed at least in part by the sheath, wherein one fiber optic cable is operative for delivering the electromagnetic waves to the sensor material and the other fiber optic cable is operative for delivering the diffracted electromagnetic waves to the means for determining.

4. The system of claim 2, wherein the at least one fiber optic cable delivers the electromagnetic waves to the sensor material and delivers the diffracted electromagnetic waves to the means for determining.

5. The system of claim 1, wherein the electromagnetic waves comprise visible light.

6. The system of claim 1, wherein:
the sensor material is comprised of either polymerized crystalline colloidal array (PCCA) material or holographic material; and
the PCCA material and the holographic material are each comprised of a hydrogel, means for diffraction and a molecular recognition agent that expands and contracts the sensor material as a function of the amount of glucose in contact therewith.

7. The system of claim 6, wherein:
the hydrogel of the PCCA material is one of the following: an acrylamide; purified agarose; N-vinylpyrolidone; methacrylate or hydroxy-ethyl-methacrylate; and
the hydrogel of the holographic material is one of the following: polyvinyl alcohol; polyvinylpyrrolidone; polyhydroxyethyl acrylate; polyhydroxyethyl methacrylate; polyacrylamides; polymethacrylamides; homopolymers, or copolymers, of polymerisable derivatives of crown ethers; or esters of, or co- or terpolymers of, polyhydroxyethylacrylate, polyhydroxyethylmethacrylate, polymethacrylamide or polyacrylamide.

8. The system of claim 6, wherein the PCCA material is either acrylamide or hydroxy-ethyl-methacrylate.

9. The system of claim 6, wherein:
the means for diffraction of the PCCA material is one of the following: self organized colloidal particles or charged colloidal particles selected from the group consisting of colloidal polystyrene, polymethylmethacrylate, silicon dioxide, aluminum oxide, polytetrafluoroethylene and poly N-isopropylacrylamide; and
the means for diffraction of the holographic material is silver halide.

10. The system of claim 6, wherein:
the molecular recognition agent of the PCCA material is one of the following: glucose oxidase; dihydroxides of boron, barium, calcium, magnesium, and strontium; boronic acid; phenylboronic acid; p-nitrophenylboronic acid; 4-methoxyphenylboronic acid; alpha-naphthylboronic acid; 4-aminomethyl-2-N,N′-dimethylaminomethylphenylboronic acid; 3-fluoro-4-aminophenylboronic acid; 2-fluoro-5-aminophenylboronic acid, or boronic acid derivatives having a pKₐ value less than 7; and
the molecular recognition agent of the holographic material is one of the following boronic acids:
2-(((4-(acrylamidomethyl)phenylamino)methyl)phenylboronic acid;
2-((3-methacrylamidopropylamino)methyl)phenylboronic acid;
acrylamido-phenylboronic acid;
2-acrylamido-phenylboronic acid;
3-acrylamido-phenylboronic acid;
3-acrylamido-6-fluoro-phenylboronic acid;
amino-fluoro-phenylboronic acid;
4-amino 3-fluoro-phenylboronic acid; or
5-amino 2-fluoro-phenylboronic acid.

11. The system of claim 1, wherein the means for determining is operative for periodically determining a concentration of glucose in the blood.

12. The system of claim 1, wherein the sheath is made from a polymer having a coating that avoids the growth of microorganisms.

13. A method of measuring blood glucose concentration comprising:
(a) inserting a vascular access catheter percutaneously into a blood vessel;
(b) inserting through the lumen of the catheter and into a flow of blood in the blood vessel at least the distal end of an optrode comprised of at least one fiber optic cable, a biocompatible sheath enclosing at least part of the at least one fiber optic cable, sensor material supported at the distal end of the sheath in optical communication with one end of the at least one fiber optic cable, the sensor material operative for diffracting electromagnetic waves received from the at least one fiber optic cable, the sensor material responsive to changes in an amount of glucose in contact therewith for changing a diffraction of the electromagnetic waves as a function thereof, and a biocompatible membrane covering the distal end of the sheath, the biocompatible membrane in contact with the sensor material and operative for passing to the sensor material glucose from blood in contact with the side of the membrane opposite the sensor material while avoiding the passage of one or more blood proteins to the sensor material;
(c) inputting electromagnetic waves into the at least one fiber optic cable;
(d) estimating from the diffraction of the electromagnetic waves input in step (c) a concentration of glucose in the blood; and
(e) outputting a signal related to the estimate in step (d).

14. The method of claim 13, wherein steps (c) and (e) are repeated substantially continuously.

15. The method of claim 13, wherein the electromagnetic waves of step (c) comprise visible white light.

16. The method of claim 13, wherein the optrode includes another fiber optic cable enclosed at least in part by the sheath, wherein one fiber optic cable is operative for delivering the electromagnetic waves to the sensor material and the other fiber optic cable is operative for delivering the diffracted electromagnetic waves from the sensor material.

17. The method of claim 13, wherein:
the sensor material is comprised of either polymerized crystalline colloidal array (PCCA) material or holographic material; and
the PCCA material and the holographic material are each comprised of a hydrogel, means for diffraction and a molecular recognition agent that expands and contracts the sensor material as a function of the amount of glucose in contact therewith.
